(19) United States
(12) Patent Application Publication
(10) Pub. No.: US 2005/0222273 A1
(43) Pub. Date: Oct. 6, 2005

(54) Treatment of female sexual dysfunction

(76) Inventor: George Dodd, Wester Ross (GB)

Correspondence Address: NIXON & VANDERHYE, PC, 901 NORTH GLEBE ROAD, 11TH FLOOR, ARLINGTON, VA, 22203, US

(21) Appl. No.: 10/505,429

(22) PCT Filed: Feb. 28, 2003
(86) PCT No.: PCT/GB03/00843

§ 371 Date: May 12, 2005

(30) Foreign Application Priority Data
Mar. 1, 2002 (GB) 0204919.5

(51) Int. Cl. A61K 31/12; A61K 31/11; A61K 31/075

(52) U.S. Cl. 514/681; 514/699; 514/718

(57) ABSTRACT

A treatment for female sexual dysfunction by the provision of at least one dopamine mimetic odorant, the odorant being capable of causing a physiological effect in, or from, the human brain which ameliorates the dysfunction, particularly Hypoactive Sexual Desire Disorder (HSDD), and delivery systems therefore.

The present invention relates to the treatment of female sexual dysfunction and is directed particularly, but not exclusively, to Hypoactive Sexual Desire Disorder.

The term female sexual dysfunction is a term used for various disorders of the sexual process in women and is categorised by pain associated with intercourse or by a disturbance in the process that forms the sexual response cycle. The sexual response cycle is divided into four distinct phases consisting of (i) the desire phase; (ii) the arousal or excitement phase; (iii) the orgasmic or plateau phase; and (iv) the resolution phase.

The (i) desire phase involves fantasies about and the desire to have sexual activity. The lack of such fantasies and desires and the general lack of receptivity to sexual activity is referred to as Hypoactive Sexual Desire Disorder (HSDD). This disorder may cause personal distress or interpersonal difficulties.

The (ii) arousal or excitement phase is typically evidenced by vaginal lubrication as a consequence of increased fluid production through blood flow. Dysfunction in this cycle is termed Sexual Arousal Disorder and manifests itself as an inability to obtain or maintain sufficient and adequate vaginal lubrication or a swelling response during this phase. This disorder is commonly associated with post-menopausal women and may cause extreme personal distress and interpersonal difficulties. That said, all women may be affected by this disorder at some stage in their lives.

The final phase, (iv) the resolution phase, is when the blood flow returns to normal and muscle tension and tightness dramatically decrease.

There are further forms of sexual dysfunction, such as female orgasmic disorder which is described as a persistent or recurrent delay or absence of an orgasm following the normal sexual excitement phase. This disorder may be present in women as a result of surgery or hormone deficiencies and/or present in women who have never achieved an orgasm.

Additionally, there are sexual pain disorders and these generally fall into one of two categories. The first category is dyspareunia and concerns genital pain associated with sexual intercourse. Pain can be associated with initial penetration or during deep thrusting, resulting in an intensity of pain which makes the prolonging of intercourse near impossible. The other category of sexual pain disorder is vaginismus and this is defined as the recurrence or persistent involuntary spasm or contraction of the perineal muscles that surround the outer third of the vagina when the insertion of any object is attempted.

The female sexual dysfunction which is of principal concern to the present invention is the treatment of Hypoactive Sexual Desire Disorder (HSDD). Of all female sexual problems, a lack of interest in sexual activity is most prevalent, affecting 30% of women.¹
¹ Laumann E O, Paik A, Rosen R C. Sexual dysfunction in the United States. JAMA 1999; 281: 537-544.

HSDD concerns sexual desire and this desire has two main manifestations—“proceptivity” (seek out and/or initiate sexual activity) and “receptivity” (accept sexual activity when offered). These manifestations are analogous to an appetite for food, the analogy being that when we feel hungry we seek out food, or when we do not feel hungry we do not seek out food until we see it tasteful and attractively presented. For many women, the complaint of lack of sexual desire is found, typically, to mean a lack of proceptivity, the woman maintaining receptive sexual behaviour.

Indeed the “appetite” phase of sexual functioning can be conceptualised in two components—designated “sexual drive” and “sexual desire”.² In this dichotomisation, the term “sexual drive” means the biological drive that generates proceptivity and receptivity. This drive is omnipotent in that it can be satisfied by a variety of behaviour (e.g. sexual activity with a partner, masturbation, sexual fantasies). Conversely, the term “sexual desire” is used to mean a desire for a specific behaviour. In effect, “sexual desire” is focused sexual drive.
² Riley A. Problems of the sexual response cycle. The Diplomate, The Journal of the Diplomats of the Royal College of Obstetricians and Gynascologists, 1997; 4: 270-275.

It will be appreciated that the physiology of sexual drive in women is not well understood. Sexual drive is thought to be generated in specific nuclei of the hypothalamus, often called collectively the “sex drive centre”. These nuclei have positive (sexually enhanced) and negative (sexually inhibiting) influences from other centres of the central nervous system mediated through neurotransmitters and neurohormones.

Treatment of HSDD may be two fold. Firstly and importantly, a correct diagnosis of HSDD would need to be made. Once a physician is comfortable that there is no organic cause and that any severe medical conditions, such as diabetes or heart disease, were correctly treated, it would then be necessary to discuss interpersonal difficulty with a partner. Once HSDD is diagnosed, the condition would be amiable to treatment with sexual therapy. A further treatment may be by hormone therapy.

Oestrogen therapy is commonly used in the pharmacological treatment of sexual dysfunction in women. Oestrogen-based therapies are generally used to increase mucus production, to provide vasodilatory effects, or to increase the general health of the vagina.³ In such treatments, oestrogen is administered orally, by injection or topically. With oral administration, the oestrogen concentration encountered by the liver is generally four to five times greater than the oestrogen levels in peripheral blood. This effect may lead to an undesirable increase in the production of certain coagulation factors and renin substrates in the liver. Oestrogen administered by injection avoids this effect in the liver. However, all oestrogen-based therapies are known to increase the risk of endometrial hyperplasia, endometrial cancer and breast cancer in treated individuals. Due to the increased risk of endometrial hyperplasia and endometrial cancer encountered with oestrogen therapies, the use of oestrogen/progestogen combinations have been utilised. However, these have the common side effects of inducing uterine bleeding and the continuation of menstrual periods.
³ Nadelson et al., eds., Treatment Inverventions in Human Sexuality (New York: Plenum Press, 1983)

Alternatively, male hormones collectively known as androgens, such as testosterone, and the lack of these in the female body have been linked to forms of sexual dysfunction. Testosterone is produced in a woman's body and the pinnacle of production is generally around the woman's reproductive years. This production can wane for a variety of reasons and this waning can be counter-acted in certain instances by testosterone replacement therapy. In certain cases this counter-acting effect has prompted an increase in sex drive.

Nevertheless, testosterone replacement therapy carries with it several dangers analogous to oestrogen replacement therapy and as such is far from satisfactory.

Accordingly, it is a general aim of the present invention to provide a treatment for female sexual dysfunction and delivery method for said treatment, and specifically the treatment of HSDD, which overcomes or ameliorates the problems and drawbacks associated with the prior art as well as, preferably, overcoming other apparent problems and drawbacks with medicaments for female sexual dysfunction in general.

According to a first aspect of the present invention, there is provided an aroma in the form of at least one dopamine mimetic odorant wherein said odorant(s) is capable of causing a physiological effect in, or from, the human brain which causes the amelioration of female sexual dysfunction, and particularly the amelioration of HSDD.

The use of an aroma is advantageous since it is capable of acting directly on the brain without having to pass through the blood brain barrier, this is in stark contrast to medicaments which are typically administered by ingestion or intravenously.

The dopamine mimetic ordorants (DMO) of the present invention are designed such that they can be sensed by a patient's olfactory system and act as a stimuli, or binder, to the patient's receptor proteins in the olfactory tract of the patient's brain to cause the amelioration of female sexual dysfunction, and particularly the amelioration of HSDD.

Whilst the exact mechanism is far from clear, it is suspected that DMOs of the present invention act in such a way as to initiate a chain of co-ordinated events in the brain which ultimately lead to changes in the patient's levels of dopamine and, consequently, the required amelioration. However, it is to be understood that the DMOs of the present invention may be working quite differently than has been hypothesised and that the amelioration is achieved by means other than changing a patient's level of dopamine in the brain.

It is to be understood that the use of the word dopamine here is intended to include other related neurotransmitters such as epinephrine, norpinephrine and the like.

The DMOs of the present invention are provided in a form capable of being sensed to a patient's olfactory system and comprises at least one of the following groups of compounds: steroid odorants; amines and related molecules; heterocyclic aroma molecules, including Jasmin oil; aromatic molecular mimics of the central neurotransmitter dopamine; carboxylic acids including those which may occur in trace levels in human secretions; aldehydes, including those which form trace ingredients in odorous human secretions.

Preferably, the DMOs of the present invention comprise at least one of the following recognized classes of perfume odorants:—vanilla like—(prototypical example—vanillin); carnation like—(prototypical example—isoeugenol); musk like—(prototypical example—6,7-dihydro-1,1,2,3,3-pentamethyl-4(5H)-indanone).

Most preferably, the DMOs of the present invention comprise at least one of the following groups of compounds: vanillin; isoeugenol and a compound in the musk category, preferably the woody musk category.

Ideally, the DMOs of the present invention comprise at least one of the following: ethyl vanillin (3-ethoxy-4-hydroxybenzaldehyde); isoeugenol (2-methodoxy-4-(1-propenyl) phenol; the woody musk 6,7-dihydro-1,1,2,3,3-pentamethyl-4(5H)-indanone.

Desirably said carboxylic acids exhibit paradoxical aroma profiles, wherein these profiles combine reassuring odour notes of warm human flesh but wherein said odour notes are embedded in distinctly fruity odour notes, these odour notes can often serve to prevent the conscious mind apprehending the occult, erogenic odour note. Furthermore said aldehydes may additionally comprise new synthetic aldehydes which can exhibit a floral odour note with a heart of erogenicity.

According to a second aspect of the present invention, there is provided a range of compound(s) in the form of at least one dopamine mimetic odorant, wherein said odorant(s) is capable of causing a physiological effect in, or from, the human brain which causes the amelioration of female sexual dysfunction, and particularly ameliorate the effects of HSDD.

According to a third aspect of the present invention, there is provided a medicament comprising a range of compounds including at least one dopamine mimetic odorant wherein said odorant(s) is capable of causing a physiological effect in, or from, the human brain which causes the amelioration of female sexual dysfunction, and particularly the amelioration of HSDD.

According to a fourth aspect of the present invention, there is provided a set of formulations comprising at least one dopamine mimetic odorant, wherein said odorant(s) is capable of effecting a physiological effect in, or from, the human brain which causes the amelioration of female sexual dysfunction, and particularly the amelioration of HSDD.

According to a fifth aspect of the present invention, there is provided a medicament comprising a set of target formulations wherein said formulations include at least one dopamine mimetic odorant and said odorant(s) is capable of effecting a physiological effect in, or from, the human brain which causes the amelioration of female sexual disorder, and particularly the amelioration of HSDD.

According to a sixth aspect of the present invention, there is provided a medicament for application to a patient in avoidance of the human blood brain barrier, wherein said medicament comprises at least one dopamine mimetic odorant and said odorant(s) is capable of effecting a physiological effect in or from the human brain which causes the amelioration of female sexual disorder, and particularly the amelioration of HSDD.

According to a seventh aspect of the present invention, there is provided a compound or compounds possessing at least one dopamine mimetic odorant wherein said odorant(s) has an appropriate molecular size and electrical charge to interact with a neuroreceptor in the human brain in such a way as to have an agonistic effect in order to cause the emission of neurotransmitters which cause an effect to the central nervous system, said effect being a physiological effect which causes the amelioration of female sexual disorder, and particularly the amelioration of HSDD.

According to an eighth aspect of the present invention, there is provided a formulation or formulations possessing at least one dopamine mimetic odorant wherein said odorant(s) is capable of interacting with a neuroreceptor in such a way as to have an agonistic effect in order to cause the emission of neurotransmitters which cause and effect to the central nervous system, said effect being a physiological effect which causes the amelioration of female sexual disorder, and particularly the amelioration of HSDD.

According to a ninth aspect of the present invention, there is provided a dopaminergic agonist(s) in the form of at least one dopamine mimetic odorant, wherein said odorant(s) is capable of causing a physiological effect in or from the human brain to cause the amelioration of female sexual disorder, and particularly the amelioration of HSDD.

According to a tenth aspect of the present invention, there is provided a natural human odorant comprising at least one dopamine mimetic odorant(s) and/or a synthetic human odorant comprising at least one dopamine mimetic odorant(s) either or both of which are capable of causing a physiological effect in or from the human brain which causes the amelioration of female sexual dysfunction, and particularly the amelioration of HSDD.

According to an eleventh aspect of the present invention, there is provided a medicament in the form of an aroma comprising at least one dopamine mimetic odorant(s) wherein said aroma has been masked or disguised such that the at least one dopamine mimetic odorant(s) contained therein is not immediately apparent on application to the patient, yet capable of causing a physiological effect in or from the human brain which causes the amelioration of female sexual disorder, and particularly the amelioration of HSDD.

According to a twelfth aspect of the present invention, there is provided an aroma which has a predetermined character as defined by an electronic nose comprising at least one dopamine mimetic odorant, wherein said aroma is capable of causing a physiological effect in or from the human brain which causes the amelioration of female sexual dysfunction, and particularly the amelioration of HSDD.

The at least one dopamine mimetic odorant may be provided in a suitable solution, such as ethanol, in a concentration up to 40% by volume. Ideally, the concentration is up to 20% by volume. The concentration of the at least one dopamine mimetic odorant can vary depending upon which DMO or DMOs are being used and the delivery means employed.

The aforementioned aspects of the present invention describe the DMOs of the present invention. The aspects of the invention mentioned hereinafter describe the delivery systems of the present invention for administration of the DMOs to a patient.

According to a thirteenth aspect of the present invention, there is provided a delivery system for any of the at least one dopamine mimetic odorant of the present invention to the patient in the form of a nasal spray. The nasal spray may be administered to the patient by a medical practitioner or by the patient themselves. The at least one dopamine mimetic odorant may be present in as a fluid in an appropriate concentration to be administered to the patient by the form of said nasal spray. Said nasal spray can comprise a reservoir in operative connection with a pump action spray nozzle. The fluid can be housed in a reservoir of the nasal spray until it is to be administered to a patient. It is preferably envisaged that the nasal spray will be placed in or near the nasal cavity/cavities of the patient to facilitate administration of the at least one dopamine mimetic odorant.

According to a fourteenth aspect of the present invention, there is provided delivery system in the form of a patch which is to be worn by the patient which is coated or impregnated with the at least one dopamine mimetic odorant. Typically, the patch is adhered to the skin of the patient to allow the at least one dopamine mimetic odorant to be emitted therefrom and into the patient via their nasal cavities. Such patches are available from The Aromacology Patch Company.

Alternatively, the patch or a strip of patch material may be housed in a protective sheath, such as a plastic sheath, to allow the insertion of same into the nasal cavities of the patient, such that the patient may draw the active ingredient of the present invention into their nasal cavities via an inhalation of breath through their nasal cavities. It is to be appreciated that the appropriate concentrations of the at least one dopamine mimetic odorant are to be used in the patch delivery system and may be included with any other suitable agent to facilitate the release of said at least one dopamine mimetic odorant into the nasal cavity/cavities of the patient.

According to a fifteenth aspect of the present invention, there is provided a delivery system for the at least one dopamine mimetic odorant of the present invention which may be applied topically to the patient for inhalation via the nasal cavities of the patient. Such topical applications could be provided in the form of a lipstick or perfume or any other suitable means which are to be applied to the skin and which are capable of being inhaled by the patient via their nasal cavities.

In order to allow the present invention to be more readily understood embodiments are described below by way of example.

The most preferred DMOs of the present invention comprise at least one of the following: ethyl vanillin (3-ethoxy-4-hydroxybenzaldehyde); isoeugenol (2-methodoxy-4-(1-propenyl) phenol; the woody musk 6,7-dihydro-1,1,2,3,3-pentamethyl-4(5H)-indanone. In order to determine their effectiveness against non-DMOs, a series of formulations were prepared for testing against a series of standardised control formulations.

The DMO formulations comprised (all in ethanol solution to 100% by volume):
1. 20% Ethyl vanillin (EV)
2. 20% Ethyl vanillin (EV) and Isoeugenol (4:1 ratio)
3. 20% Ethyl vanillin (EV) and Isoeugenol and woody musk 6,7-dihydro-1,1,2,3,3-pentamethyl-4(5H)-indanone
4. 20% woody musk 6,7-dihydro-1,1,2,3,3-pentamethyl-4(5H)-indanone

The standardised control formulations comprised (all in ethanol solution to 100% by volume):
5. Natural animal cologne
6. Strong carnation natural perfume
7. Intensely sweet fruity perfume

The formulations were applied to separate strips of material and the testers were requested to grade each odour produced on a five-point scale (A-E). The A end of the spectrum denoting “Like very much/including arousal” and E at the other end of the spectrum “Do not like very much”.

Before the experimentation it was hypothesised that the standardised control formulations would produce the following response
5. Natural animal cologne would produce a significant negative response, mainly E and D responses.
6. Strong carnation natural perfume would produce a response roughly in the middle of the spectrum, mainly C responses.
7. Intensely sweet fruity perfume would produce a significant negative response, mainly E and D responses.
   Results

The following represent the % of testers grading the odour is marked.
1. Majority A or B
2. Majority A or B
3. Majority B or C
4. Majority A
5. Majority C, D or E
6. Majority B, C or D
7. Majority E

Due to numerous inherent limitations in performing tests of this nature, one such limitation being adaptation when forced to test numerous odours in a short space of time, as well as other well known limitations, it is interesting to note that the testers graded the standardised control formulations (5-8) as expected. More interesting however, is that the formulations of the present invention (1-4) all obtained a majority of the A end of the spectrum, denoting “Like very much/including arousal”.

Of course numerous modifications and variations are envisaged with the formulations, compounds, aromas, medicaments or dopamenic agonists of the present invention and these will be apparent to the skilled person without need to depart from the invention.

1-19. (canceled)

20. A formulation comprising a vanilla like odorant, a carnation like odorant and a musk like odorant.

21. A formulation according to claim 20 comprising
up to 40% (by volume) of a vanilla like odorant
up to 40% (by volume) of a carnation like odorant
up to 40% (by volume) of a musk like odorant

22. A formulation according to claim 20 comprising
up to 20% (by volume) of a vanilla like odorant
up to 20% (by volume) of a carnation like odorant
up to 20% (by volume) of a musk like odorant

23. A formulation according to claim 20 wherein the vanilla like odorant is a vanillin.

24. A formulation according to claim 20 wherein the vanillin is ethyl vanillin.

25. A formulation according to claim 20 wherein the carnation like odorant is isoeugenol.

26. A formulation according to claim 20 wherein the musk like odorant is a woody musk odorant.

27. A formulation according to claim 26 wherein the woody musk odorant is 6,7-dihydro-1,1,2,3,3-pentamethyl-4(5H)-indanone

28. A formulation according to claim 20 comprising
1-19% (by volume) 3-ethoxy-4-hydroxybenzaldehyde
up to 5% (by volume) 2-methoxy-4-(1-propenyl) phenol
1-19% (by volume) 6,7-dihydro-1, 1,2,3,3-pentamethyl-4(5H)-indanone

29. A formulation according to claim 20 further comprising a carboxylic acid.

30. A formulation according to claim 29, which exhibits paradoxical aroma profiles.

31. A formulation according to claim 30 wherein the paradoxical aroma profiles combine odour notes of warm human flesh embedded in fruity odour notes.

32. A formulation according to claim 20 further comprising synthetic aldehydes, which exhibit a floral odour note with a heart of erogenicity.

33. A formulation substantially as herein described.

34. A skin patch comprising a formulation according to claim 20.

35. A nasal spray comprising a formulation according to claim 20

36. A lipstick comprising a formulation according to claim 20.

37. A perfume comprising a formulation according to claim 20.

38. A delivery device in the form of a strip of material housed in a protective sheath coated or impregnated with a formulation according to claim 20.

39. A delivery device according to claim 38, wherein the protective sheath is provided with suitable closure means to control the release of the formulation from the sheath.

40. The use of a formulation according to claim 20 for the treatment of female sexual dysfunction.

41. A method of treating female sexual dysfunction comprising administering to the olfactory system of a patient, an effective amount of the formulation disclosed in claim 20.

42. A method of treating female sexual dysfunction according to claim 41 wherein the formulation is administered to the patient by application to the patients skin of a patch, which is coated or impregnated with the formulation.

43. A formulation according to claim 20 for use in the treatment of female sexual dysfunction.

44. The use of a formulation according to claim 20 in the production of a medicament of the treatment of female sexual dysfunctions.
